(19) United States
(12) Patent Application Publication
(10) Pub. No.: US 2024/0177819 A1
(43) Pub. Date: May 30, 2024

(54) STORAGE MEDIUM STORING OPHTHALMOLOGY INFORMATION PROCESSING PROGRAM, OPHTHALMOLOGY COMPUTER AND OPHTHALMOLOGY INFORMATION PROCESSING METHOD

(71) Applicant: NIDEK CO., LTD., Gamagori-shi (JP)

(72) Inventors: Seiichirou SHIMIZU, Gamagori-shi (JP); Haruka UEMURA, Gamagori-shi (JP); Maki TSUKUDA, Gamagori-shi (JP); Masanori NAKANO, Gamagori-shi (JP); Tetsuya KANO, Gamagori-shi (JP); Norimasa SATAKE, Gamagori-shi (JP)

(21) Appl. No.: 18/431,484

(22) Filed: Feb. 2, 2024

Related U.S. Application Data
(63) Continuation of application No. PCT/JP2022/023060, filed on Jun. 8, 2022.

(30) Foreign Application Priority Data
Aug. 4, 2021 (JP) 2021-128629
Sep. 30, 2021 (JP) 2021-162175

(51) Int. Cl. G16H 15/00

(52) U.S. Cl. CPC G16H 15/00

(57) ABSTRACT

A non-transitory, computer readable storage medium stores an ophthalmology information processing program for at least one processor of an ophthalmology computer. The program includes instructions, when executed by the at least one processor, cause the at least one processor to: sequentially display a plurality of examination results on a screen; generate a plurality of converted examination results by converting the plurality of examination results in a predetermined format; and collectively performing a transfer process on the plurality of converted examination results each corresponding to a respective one of the plurality of examination results upon receiving a transfer operation. When generating the plurality of converted examination results, the instructions further cause the at least one processor to generate at least one of the plurality of converted examination results before receiving the transfer operation.

### CROSS REFERENCE TO RELATED APPLICATION

This application is a continuation application of International Patent Application No. PCT/JP2022/023060 filed on Jun. 8, 2022, which designated the U.S. and claims the benefit of priority from Japanese Patent Application No. 2021-128629 filed on Aug. 4, 2021 and Japanese Patent Application No. 2021-162175 filed on Sep. 30, 2021. The entire disclosure of the above application is incorporated herein by reference.

### TECHNICAL FIELD

The present disclosure relates to a storage medium storing an ophthalmology information processing program, an ophthalmology computer and an ophthalmology information processing method.

### BACKGROUND

The examination reports are utilized during diagnosis at ophthalmological facilities. In the examination report, images, measured values, their analysis results, and the like acquired through an eye examination device are shown in a predetermined format as an examination result.

For example, the examination report is created by an examiner who conducted the examination. Then, the diagnosis using the examination report may be made by another person. For example, such examination reports created by examiners are managed in another system such as a medical information management system in a facility. This allows examination reports to be viewed via a terminal device in the system.

### SUMMARY

In one aspect of the present disclosure, a non-transitory, computer readable storage medium stores an ophthalmology information processing program for at least one processor of an ophthalmology computer. The program includes instructions, when executed by the at least one processor, cause the at least one processor to: sequentially display a plurality of examination results on a screen, the plurality of examination results being examination results on a subject's eye obtained by an eye examination device; generate a plurality of converted examination results by converting the plurality of examination results in a predetermined format; and collectively perform a transfer process on the plurality of converted examination results each corresponding to a respective one of the plurality of examination results upon receiving a transfer operation. When generating the plurality of converted examination results, the instructions further cause the at least one processor to generate at least one of the plurality of converted examination results before receiving the transfer operation.

### BRIEF DESCRIPTION OF DRAWING

FIG. 1 is a schematic configuration diagram illustrating an ophthalmologic system according to first and second embodiments.

FIG. 2 is a flowchart showing a flow of an operation in the first embodiment.

FIG. 3 is a flowchart showing a flow of an operation according to the second embodiment.

FIG. 4 is a diagram showing an examination result selection screen image according to an embodiment.

FIG. 5 is a diagram showing an examination result display screen image according to an embodiment.

### DESCRIPTION OF EMBODIMENTS

Next, a relevant technology will be described first only for understanding the following embodiments.

Conventionally, when transferring examination results such as reports converted into a predetermined format to another system, an examiner needed to check the examination results one by one and corrected their contents as necessary. Alternatively, it was assumed that an examiner conducted a re-examination. For this reason, each examination result was displayed for the examiner to confirm, and each examination result was not automatically transferred to another system until a transfer operation was made by the examiner. Furthermore, multiple examination results could not be transferred at once only by a single transfer operation. Since there were such constraints, it was assumed that the above-described operation would work.

However, with improvements in examination techniques (for example, equipment alignment techniques, analysis techniques, etc.), inappropriate examination results are less likely to be obtained recently. For this reason, it would cause an excessive burden on an examiner if the examiner still needs to check the examination results one by one.

In view of the above, one of objectives of the present disclosure is to reduce the burden on examiners when transferring reports to another system.

In a first aspect of the present disclosure, a non-transitory, computer readable storage medium stores an ophthalmology information processing program for at least one processor of an ophthalmology computer. The program includes instructions, when executed by the at least one processor, cause the at least one processor to: sequentially display a plurality of examination results on a screen, the plurality of examination results being examination results on a subject's eye obtained by an eye examination device; generate a plurality of converted examination results by converting the plurality of examination results in a predetermined format; and collectively perform a transfer process on the plurality of converted examination results each corresponding to a respective one of the plurality of examination results upon receiving a transfer operation. When generating the plurality of converted examination results, the instructions further cause the at least one processor to generate at least one of the plurality of converted examination results before receiving the transfer operation.

In a second aspect of the present disclosure, an ophthalmology computer includes: at least one processor; and at least one memory storing an ophthalmology information processing program. The at least one processor causes, by executing the program, the ophthalmology computer to: sequentially display a plurality of examination results on a screen, the plurality of examination results being examination results of a subject's eye obtained by an eye examination device; generate a plurality of converted examination results by converting the plurality of examination results in a predetermined format; and collectively perform a transfer process on the plurality of converted examination results each corresponding to a respective one of the plurality of examination results upon receiving a transfer operation. When generating the plurality of converted examination results, the at least one processor further causes the ophthalmology computer to generate at least one of the plurality of converted examination results before receiving the transfer operation.

In a third aspect of the present disclosure, a method implemented by an ophthalmology computer includes: sequentially displaying a plurality of examination results on a screen, the plurality of examination results being examination results of a subject's eye obtained by an eye examination device; generating a plurality of converted examination results by converting the plurality of examination results in a predetermined format; and collectively performing a transfer process on the plurality of converted examination results each corresponding to a respective one of the plurality of examination results upon receiving a transfer operation. When generating the plurality of converted examination results, the method further comprises generating at least one of the plurality of converted examination results before receiving the transfer operation.

Next, a plurality of embodiments of the present disclosure will be described below. It should be noted that the items classified in < > below can be used independently or in conjunction with each other. Further, part or all description in an embodiment may be also used in other embodiments.

### <Device Configuration in Embodiment>

An ophthalmology information processing program according to each of the following embodiments in the present disclosure is executed by at least one processor of an ophthalmology computer. The ophthalmology computer may be integrated with an eye examination device.

The ophthalmology computer is connected to a medical information system. The medical information system stores and manages examination results obtained by various examination devices. For example, examination reports created by an ophthalmology computer are managed by a medical information system.

The eye examination device has an examination unit (a measuring unit or an imaging unit) used for examination of subject eyes. The eye examination device may be a measuring device that measures subject eyes or may be an imaging device that captures images of the subject eyes. Examples of the measuring device include a refractive power measuring device, aberrometers, an ocular axis length measuring device, an intraocular pressure measuring device, a perimeter, and the like. The eye examination device may be an imaging device that captures images of subject eyes and obtains the images of the subject eyes as examination results. Examples of the imaging device include an OCT device (an optical coherence tomography), a fundus camera, an SLO device, and so on. Note that by processing a received-light signal obtained by the imaging device, the measurement result of the subject eye may be obtained as a examination result. For example, by processing OCT data or OCT images obtained by an OCT device (an optical coherence tomography), measurement results relating to tissue layer thickness, size, shape, blood vessels, etc. may be obtained. However, the eye examination device illustrated herein is only one example, and other devices may be used. Furthermore, the eye examination device may be an integrated device that is formed of a combination of measurement devices and imaging devices.

### <Schematic Description of Operation>

In each embodiment of the present disclosure, the ophthalmology computer obtains examination results of subject eyes by the eye examination device (an obtaining step). Further, the ophthalmology computer generates a converted examination result that is formed by converting the acquired examination result into a predetermined format (a generation step). In this case, the predetermined format may be the form of an examination report. The examination report may be created based on an examination result and a pre-prepared template. In the template, at least the content (combination) and arrangement of items of information that are arranged on the examination report may be defined. The examination report is used, for example, for diagnosis by a doctor. Here, when an image of the subject eye is acquired as an examination result and an examination report is created, the ophthalmology computer may process the image of the subject eye and create the examination report including the image of the subject eye. For example, the examination report may include at least an image of a part of the subject eye for the purpose of the examination report. Additionally, the examination report may further include at least one of a measurement result and an analysis result on the image of the subject eye.

Further, when an image of the subject eye is acquired as an examination result, the converted examination result may be formed in DICOM format.

In each embodiment, the ophthalmology computer executes a transfer process on the generated converted examination result (a transfer step). Based on the transfer process, for example, the converted examination results are transferred to a medical information system. The medical information system, for example, digitizes medical information and collectively manages the digitized medical information at a server. At least the converted examination results are managed by the medical information system.

The transfer process may be a process of directly outputting the converted examination results from the ophthalmology computer to the medical information system. Alternatively, the transfer process may be a process where the converted examination results are temporarily stored on a memory shared between the ophthalmology computer and the medical information system, and then the medical information system is notified that the converted examination results are available for acquisition. The converted examination results are transferred as appropriate upon the medical information system accessing the converted examination results after notified.

Unless otherwise noted, the following description assumes that, during the transfer process, the converted examination results are directly output from the ophthalmology computer to the medical information system.

### <Transfer Process of Converted Examination Results in the First Embodiment

In a first embodiment of the present disclosure, converted examination results are transferred to the medical information system without requiring a transfer operation by an examiner. In the first embodiment, a first transfer step and a second transfer step are selectively performed to transfer the converted examination results to the medical information system. That is, the converted examination results are transferred via either the first transfer step or the second transfer step.

At the first transfer step, the examination result which is a base of the converted examination result is displayed and a request to an examiner for a transfer operation is displayed. For example, an examiner is requested to operate a switch that is a trigger for transfer. Note that the interface through which the transfer operation is input may be a hardware interface. Alternatively, a GUI may be used as an interface. Furthermore, at the first transfer step, the converted examination results are transferred to the medical information system when the transfer operation is received. In this manner, the first transfer step is executed on the premise that an examiner confirms the examination results before transferring the results.

At the second transfer step, the converted examination results are transferred to the medical information system without requesting the examiner for the transfer operation. That is, during the second transfer step, the transfer operation by an examiner is not a precondition for transferring the converted examination results. In this embodiment, the converted examination results are transferred without displaying a request for the transfer operation while the examination results are being displayed. Therefore, at the second transfer step, the input of the transfer operation is omitted, and therefore the burden on the examiner is reduced as compared to the first transfer step.

Furthermore, during the second transfer step, the converted examination results may be transferred before displaying the examination results. This eliminates the need for examiners to check the examination results, whereby further reducing the burden on the examiners. However, it is not necessarily required that the converted examination results be displayed by the ophthalmology computer after the converted examination results were transferred from the ophthalmology computer to the medical information system.

### <Type of Examination Reports>

When the converted examination result is used as an examination report, in this embodiment, each of the examination reports may be generated in a respective one of multiple types of templates. Each of the multiple templates is formed in association with at least one of the disease type and the examination conditions of the eye examination device. For example, the ophthalmology computer acquires information on either the type of disease in the subject eye or the examination conditions for the subject eye. Based on the information, one or more examination reports based on at least one of the multiple templates may be selectively transferred to the medical information system. Accordingly, the medical information system can selectively receive an appropriate report on which at least one of the disease type of the subject eye and the examination conditions of the subject eye is reflected. That is, diagnosis based on appropriate reports can be made.

Note that the disease type may be, for example, an automatic diagnosis result for an examination result. Further, if information specifying the disease type of a subject, such as a past diagnosis result of the subject, is stored in advance, such information can be used.

Further, for example, the followings may be specified as the examination conditions. That is, at least one of the eye(s) that was examined (i.e., either the left, right eye or both eyes), the examined area in the subject eye, and the contents (i.e., the details) of the examination may be specified as the examination conditions. For example, if the eye examination device is an OCT device, the examination site or examination details are specified based on the examination conditions such as fixation position, angle of view, scan settings (scan position, scan pattern, scan speed), and whether OCT-Angiography is used. The information indicative of the examination conditions may be supplementary information associated with the examination results. Alternatively, the information indicative of examination conditions may be the examination result itself.

As the examination report, a monocular report on which only the examination results on one eye are shown or a binocular report on which the examination results on both eyes are shown may be created. Whether to output either the monocular report or the binocular report may be determined based on at least one of first information on whether a binocular disease exists and second information on whether examination results for both left and right eyes exist. For example, it may be determined based on either information on the disease type in the subject eye or information on the examination conditions for the subject eye. Additionally, if examination results for both eyes have been obtained, the binocular report is created. On the contrary, if examination results for only one eye have been obtained, the monocular report is created. Then, each of the binocular report and the monocular report may be transferred. Accordingly, in the second transfer step, the binocular report and the monocular report can be appropriately created and transferred.

### <Automatic Selection of Eye Images to be Placed on the Examination Report>

The eye examination device is an ophthalmological imaging device, and a plurality of images of the subject eye captured at mutually different imaging positions are acquired. Furthermore, when a structural feature exists in one of the multiple images, the image of the subject eye showing this feature may be placed on the examination report. Note that the structural feature may be a feature related to a disease.

If there is a limit to the number of eye images that can be placed on the examination report, the image of the subject eye showing the feature may be placed with the first priority. For example, the images may be placed preferentially in a descending order of priority relating to the feature from the image with the highest priority. The priority may be determined according to the degree of abnormality of the structure. In particular, when the eye examination device is an OCT device and three-dimensional OCT data is obtained as one of the examination results, the degree of structural abnormality may be obtained as follows. That is, even if the degree of abnormality in a structure may be obtained based on a probability distribution obtained by inputting 3D OCT data into a trained model that is created to obtain a probability distribution for identifying tissues in an image. For more details, JP 2020-18794 A filed by the present applicant may be referred, which is incorporated by reference herein.

Furthermore, if the eye examination device is an OCT device and three-dimensional OCT data is obtained as one of the examination results, two-dimensional images of several cross-sections included in the three-dimensional OCT data may be placed on the examination report. In this case, the interval between the cross-sections may be automatically determined or manually selected depending on, for example, the number of two-dimensional images that can be placed on the report.

### <Follow-Up Examination>

In this embodiment, a follow-up examination report may be created based on examination results for the same subject's eye that were generated on different examination dates. In this case, the follow-up examination report shows changes over time between the current and past examination results. For example, both the current and past examination results and/or the differences therebetween are shown on the report.

The follow-up examination report may be created in the following cases. One of such cases is that the fact that a follow-up examination has been performed is specified from information indicative of examination conditions for the current examination or by referring to past examination results. In this case, both a report based on the current examination results and a report for the follow-up examination may be created.

### Second Embodiment

In a second embodiment, when a transfer operation is input by an examiner, a transfer process is executed on a plurality of converted examination results at once. In the second embodiment, an ophthalmology computer executes at least a selection step, a display step, a generation step, and a transfer step based on an ophthalmology information processing program.

### <Selection Step>

In the second embodiment, the selection step may be performed in order to select, from among examination results that have been obtained in advance, those for generating converted examination results. At the selection step, the plurality of examination results obtained in advance, which are examination results on a subject eye by the eye examination device, are selected at once. The plurality of examination results are put in a selected state as targets to be sequentially displayed at the display step, as will be described later. The plurality of examination results selected at once may be examination results acquired on a plurality of examination dates or may be examination results on a plurality of subjects (i.e., examinees).

### <Display Step>

The plurality of examination results are sequentially displayed on a screen, and each examination result is confirmed by the examiner. If the plurality of examination results are collectively selected at once at the selection step, the selected examination results are sequentially displayed. A switching operation by an examiner or operator may be required to sequentially switch the examination result. The switching operation may be an operation on a GUI displayed on the screen on which the examination results are sequentially displayed. Based on the switching operation, the examination result on the screen may be switched to another examination result (a switching step).

Further, an editing operation to edit an examination result may be accepted when the examination result is displayed on the screen. In this case, the ophthalmology computer changes the contents of the converted examination result corresponding to one of the examination results based on the editing operation (an editing step).

### <Transfer Step>

At the transfer step in the second embodiment, when the ophthalmology computer receives a transfer operation by an examiner, the ophthalmologic computer collectively performs a transfer process on a plurality of converted examination results each corresponding to a respective one of the plurality of examination results. At this time, the transfer process may be performed at once for at least the examination results that were displayed sequentially. As a result, the process of transferring the plurality of converted examination results to a medical information system, for example, is started by a single transfer operation as a trigger.

Note that the description of the transfer operation required at the first transfer step in the first embodiment can be applied for the transfer operation in the second embodiment.

### <Generation Step>

At the generation step, the ophthalmology computer generates a converted examination result that corresponds to the examination result. The description of the first embodiment can be applied to the contents of the converted examination result. In the second embodiment, the ophthalmology computer generates, before accepting the transfer operation, at least some of the multiple converted examination results each corresponding to a respective one of the multiple examination results that were collectively selected. That is, at least some of the multiple converted examination results are generated before the examiner receives the transfer operation (especially, while checking the examination results). The remaining of the converted examination results is generated after the transfer operation was received. By generating at least some of the multiple converted examination results before receiving the transfer operation, the necessary time from the input of the transfer operation for the multiple converted examination results until the transfer process for all the converted examination results is completed can be reduced.

The process of generating at least some of the multiple converted examination results during the generation step may be performed in the background while the examination results are being displayed on the screen. Since the process of generating the converted examination results is less likely to interfere with the sequential display of the examination results, smooth confirmation of the examination results by the examiner can be facilitated.

Further, in the second embodiment, the trigger for starting generation of the converted examination results corresponding to the examination results may be the switching operation that is input by an examiner or an operator in a state where each of the examination results is displayed on the screen. Generation of the converted examination result corresponding to the examination result that was displayed on the screen right before is started based on the switching operation. After the examiner confirmed, and sometimes edited the examination results, the switching operation is input. Therefore, even if the converted examination results for the examination results that have already been displayed are generated before the transfer operation is received, rework such as re-editing is unlikely to be required. Therefore, the converted examination results can be efficiently generated before receiving the transfer operation.

The timing at which generation of the converted examination results each corresponding to the respective examination result is started is not necessarily limited to the above-described timing. For example, while each examination result is being displayed, an examination report for the displayed examination result may be created.

### EXAMPLES

Examples according to embodiments of the present disclosure will be described with reference to the drawings.

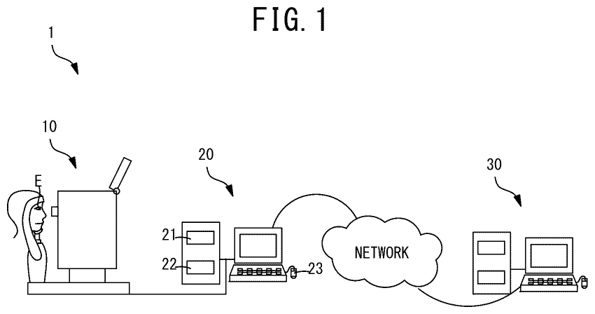

FIG. 1 shows a schematic configuration of an ophthalmologic system 1 according to the first embodiment. The ophthalmology system 1 includes an OCT device 10, a PC (personal computer) 20, and a medical information system 30. Each device may be interconnected with each other via a network.

In this embodiment, the OCT device 10 is used as an example of an eye examination device. The OCT device 10 acquires OCT data of a subject eye via an examination optical system (not shown). For example, the examination optical system includes an OCT light source, a scanning unit for scanning with OCT light, an optical system for irradiating the subject eye with OCT light, a light receiving element that receives light reflected by the tissue of the subject eye, etc. Unless otherwise specified, OCT data of the fundus is acquired in this example. For example, the OCT data may be two-dimensional OCT data (B-scan data). Alternatively, the OCT data may be three-dimensional OCT data. A two-dimensional tomographic image is generated based on the two-dimensional OCT data. Furthermore, a three-dimensional image and an OCT frontal image (may be also referred to as an “en-face image”) are acquired based on the three-dimensional OCT data. Alternatively, the OCT data may be motion contrast (MC) data. The motion contrast may be, for example, information capturing blood flow in the subject eye, changes in retinal tissue, and the like. The MC data is obtained by processing a plurality of pieces of the OCT data that were obtained at the same location of the tissue but different timings.

The OCT device 10 may further include an observation optical system. The observation optical system is an optical system different from the above-mentioned examination optical system, and obtains a front image of the fundus as an observation image. The observation optical system may be a fundus camera optical system or an SLO optical system. Other systems may be used.

In this embodiment, the PC 20 is used as an example of the ophthalmology computer. In this embodiment, the PC 20 also serves as a control unit that controls operation of the OCT device 10. Further, the PC 20 acquires, as examination results, subject eye images (i.e., various OCT images and observation images) captured via the OCT device 10, analysis results of the subject eye images, etc. Furthermore, the PC 20 creates an examination report based on the image data of the subject eye.

The PC 20 includes at least a processor 21 (a processing device) and a memory 22. In this embodiment, the following information is stored on the memory 22 in advance. A control program for the OCT device 10, an image processing program for processing the OCT data, an ophthalmological information processing program for creating and transmitting the examination report, fixed value data, and the like. Various programs are read and executed by the processor 21. Furthermore, the memory 22 may store the examination results obtained via the OCT device 10. For example, the examination results of the subject's eye obtained from past examinations may be stored on the memory 22 in advance.

An operation unit 23 such as a mouse and a keyboard may be connected to the PC 20. Various operations are input via the operation unit 23.

The medical information system 30 digitizes medical information and collectively manages the digitized medical information using a server. The managed medical information includes at least examination reports created by the PC 20. In addition, medical images, medical records, interpretation results, etc. may be managed. Note that in this embodiment, the medical image includes the OCT data acquired via the OCT device 10 and a part of the observation image. Furthermore, the medical records, the image interpretation results, and the like include disease information on subjects as subject information. Each of pieces of information is managed in association with subject's identification information.

Various types of medical information managed by the medical information system 30 can be displayed on a diagnostic terminal device (not shown). This allows for division of labor between examination and diagnosis.

### <Operation>

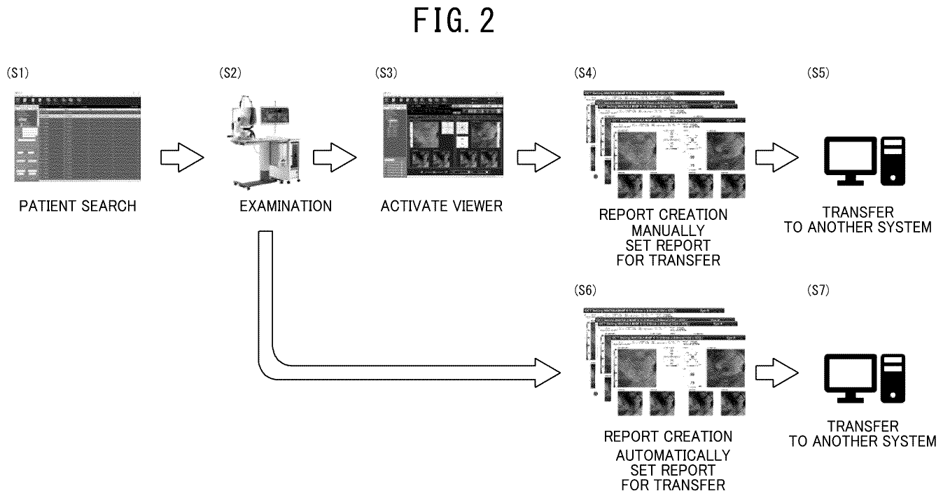

Next, with reference to the flowchart of FIG. 2, the flow of operation in the ophthalmological system 1 from examination of the subject's eye to transfer of the examination report to the medical information system 30 will be described.

First, a patient search is performed by the PC 20 (S1). By registering names and IDs of subjects in advance, a particular subject can be searched from the search screen and can be identified. As a result, the subject's information is able to be associated with examination results that are to be obtained.

Next, the PC 20 controls the OCT device 10 to examine (measure) the subject eye (S2). As a result, OCT data of the subject is acquired. At this time, the OCT device 10 may perform an imaging operation based on in accordance with conditions set by the examiner. For example, the examination conditions including at least one of the following: eye to be examined (left or right or both eyes), fixation position, angle of view, scan settings (scan position, scan pattern, scan speed), existence of OCT-Angiography, and the like may be set.

Further, a plurality of imaging operations with mutually different examination conditions may be performed. A part of the examination results may be displayed on a confirmation screen. An OCT image and an analysis result with a relatively low processing load may be displayed. If it is confirmed through the confirmation screen that an expected examination result has been obtained, this examination result is stored on the memory 22 of the PC 20. Furthermore, if it is confirmed that an unexpected examination result has been obtained (due to, e.g., poor imaging, etc.), re-imaging is performed. The confirmation screen may include a re-imaging button for inputting an instruction for re-imaging.

After the examination is completed, in this embodiment, an examination report is created and transferred. In this embodiment, the procedure branches into a manual transfer process (S3 to S5) and an automatic transfer process (S6, S7). In this embodiment, it is assumed that transfer setting indicating which transfer process is to be executed is preset based on an examiner's selection operation. Note that the transfer setting may be determined for each examination condition. For example, the transfer setting may be preset so that the automatic transfer is performed under certain examination conditions, whereas the manual transfer is performed under other examination conditions.

First, the flow of manually transferring the examination report (S3 to S5) will be explained. In this embodiment, the examination report is manually transferred via a viewer displayed by the PC 20.

First, the PC 20 activates the viewer (S3). The viewer is activated at an arbitrary timing based on an activation operation. In the viewer, the examination report is created based on examination results and a template that is prepared in advance (S4). Images based on the OCT data, analysis results of the OCT data, etc. are shown on the examination report. The analysis result may be a two-dimensional map such as a heat map. Alternatively, the analysis result may be a segmentation result. Furthermore, numerical information such as measured values may be used. The PC 20 appropriately analyzes the OCT data and obtains the analysis results.

As an example, in this embodiment, three types of reports may be created as examination reports: a macular disease report, a glaucoma report, and an angio report. Here, the macular disease report is used for diagnosing macular disease. The glaucoma report is used for diagnosing glaucoma. The angio report allows you to view the status of each vascular network. Which report is generated may be automatically set based on, e.g., the examination conditions and examination results. For example, the macular disease report may be created when the area around the macula is imaged, and the glaucoma report may be generated when a wide area of the retina is imaged. Alternatively, if MC data is acquired, the angio report may be created respectively. Further, disease information of the subject may be acquired from the medical information system 30 or the like, and an examination report may be selected according to the disease information. However, the types of reports and the contents of the imaging operation that correspond to the various reports are not necessarily limited to these.

Further, in this embodiment, a binocular report and a monocular report are selectively created. When a selection operation to select a binocular disease or a unilateral disease is received, either the binocular report or the unilateral report is created according to the selection operation.

The PC 20 automatically arranges OCT images and analysis results according to a predetermined report template. However, although the OCT device 10 captures a large number of OCT images, there is a space restriction on the number of OCT images that can be arranged on the examination report. Therefore, images that are automatically arranged may not match the examiner's intention. In view of this, the OCT images arranged on the examination report can be changed based on an operational input by the examiner.

These various examination reports are selectively displayed on the viewer. For example, a tab is provided for each of the examination reports. The examiner selects one of the tabs to switch to the desired examination report. Additionally, a transfer button image (a connecting button image) is displayed along with the examination report. By touching the transfer button image, the displayed examination report is transferred to the medical information system 30 (S5). Furthermore, if another examination report is required to be transferred, the examination report displayed on the viewer is changed and then the same operation is performed. As described above, when manually transferring the report in this way, the examiner is required to confirm the examination report each time, and then the examiner is requested to perform the transfer operation.

Next, the flow of automatically transferring the examination report (S6, S7) will be explained.

In this example, after the examination was completed, the PC 20 creates the examination report without activating the viewer (S6). Similar to the above (S4), the examination report is created based on the examination results and the template prepared in advance.

One of three types of reports (i.e., the macular disease report, the glaucoma report, and the angio report) is automatically selected based on the examination conditions and examination results. For example, whether to create one of the reports may be determined based on whether or not essential OCT images and analysis results are acquired in the template of the report.

Furthermore, in this embodiment, whether to generate the binocular report or the monocular report is determined based on the examination results obtained at the current timing and the previous timings.

For example, if the current and past examination results on only one of the left and right eyes exist, the monocular report is created. On the other hand, if the current and past examination results on each of the left and right eyes exist, the binocular report is created. When creating the binocular report, if both eyes have been examined at the current examination, the binocular report is created based on the current examination. In addition, there is a case where only one eye is examined in the current examination, and the past examination results for the other eye are available. In that case, the binocular report is created using the current examination result for one eye and the latest examination result among the past examination results for the other eye.

In this embodiment, the examination report automatically created as described above is automatically transferred to the medical information system 30 (S7). That is, in this case, it is not necessary for the examiner to check the created report on the viewer and to perform a transfer operation. Therefore, there is no need for the examiner to check each report and to input the transfer operation for each examination or report, whereby reducing the burden on the examiner. In this embodiment, the quality of the examination results is ensured by the examiner by going through the confirmation screen after the examination. As a result, the quality of the automatically created examination reports can be easily guaranteed.

### Second Embodiment

In the first embodiment, the report is created and transferred each time the examination is completed. On the contrary, in the second embodiment, after the examinations were performed on a plurality of subjects, reports are collectively transferred to the medical information system 30 at once.

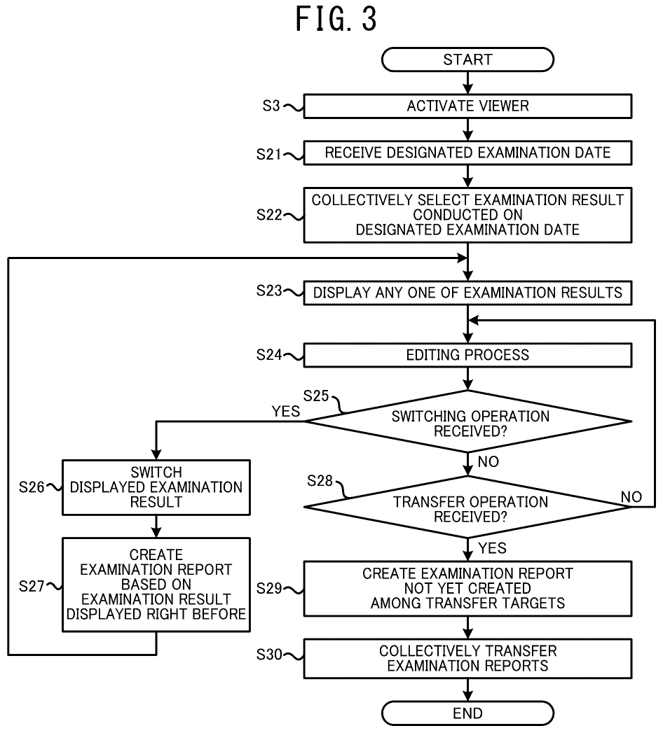

In the second embodiment, a description of the flow of the automatic transfer will be omitted, and only the manual transfer will be described. In the second embodiment, for example, all the examination reports regarding a plurality of examinations conducted on a designated examination date may be transferred at once. Hereinafter, description will be given along the flowchart of FIG. 3.

After the multiple examinations were completed, the viewer is activated (S3).

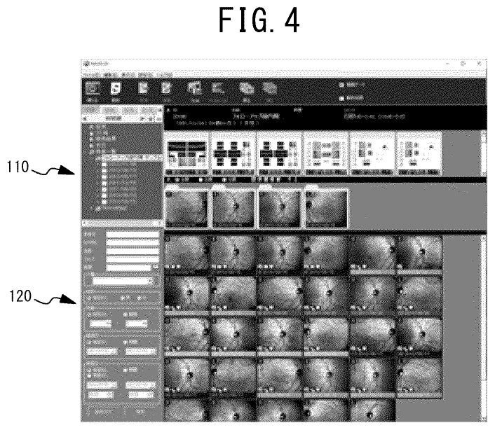

First, an examination result selection screen image shown in FIG. 4 is displayed. On the examination result selection screen image, examination results obtained in advance are shown in a list 110, for example.

A GUI 120 for setting conditions for search results to be selected is shown in the examination result selection screen image. By setting desired conditions via the GUI 120, the examination results that meet the conditions are collectively selected at once. A plurality of types of conditions for setting may be used, and an examination result may be selected by combining a plurality of conditions.

The conditions that can be set via the GUI 120 include, for example, the examination date, the subject, and the disease. For example, by specifying the examination date, a plurality of examination results that are obtained on the specified date are selected at once (S21). In the following explanation, unless otherwise specified, the examination date is set as a condition.

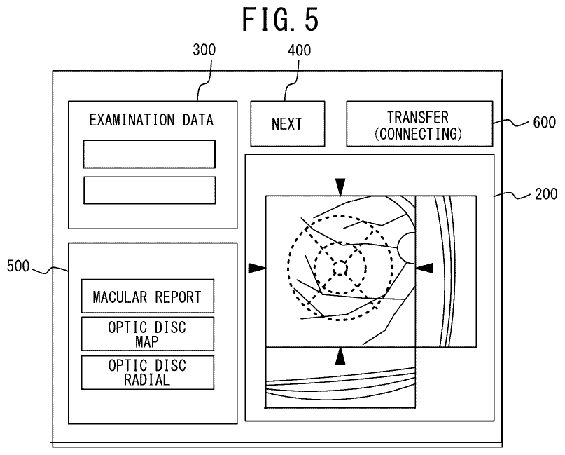

After the examination result is selected, an examination result display screen image shown in FIG. 5 is displayed. The examination results that form the basis of the examination report are confirmed by the examiner.

In this embodiment, on the examination result display screen image, one of the plurality of examination results that were collectively selected at once is selectively displayed in the display area 200 (S22). In the case of OCT, the examination results each of which is generated based on a respective scan pattern are displayed in the display area 200. Similar to the first embodiment, the PC 20 automatically arranges OCT images and analysis results for each examination according to a predetermined report template. When the OCT data for the same subject eye is acquired using multiple scan patterns in one examination, each of the scan patterns is displayed in a third list 500. The scan pattern is selected among from those displayed in the third list 500 and the scan pattern displayed in the display area 200 is switched. When the scan pattern is formed of a plurality of scan lines, a tomographic image originated from any one of the scan lines may be displayed in the display area 200. Furthermore, analysis results such as a thickness map are displayed in the display area 200.

The examination results displayed in the display area 200 are edited based on an editing operation (S24). The OCT images and the analysis results shown in the examination report can be changed based on an operational input. For example, the tomographic image displayed in the display area 200 may be changed to one generated based on another scan line. Furthermore, a reference position for analysis in the analysis results may be changed.

A second list 300 and a “next” button image 400 are displayed on the examination result display screen image. One of the plurality of examination results that were collectively selected at once and are listed In the second list 300 is displayed in the display area 200. Any one of the plurality of examination results listed in the second list 300 can be arbitrarily selected, and the selected one is displayed in the display area 200. Further, the plurality of examination results are arranged in a predetermined order (for example, in chronological order). When the “NEXT” button image 400 is touched (S25: YES), the examination result that is next in the order of the currently selected examination result is newly selected and displayed in the display area 200 (S26). Therefore, in the second embodiment, a plurality of examination results can be confirmed without changing the examination result display screen image 200, so that the confirmation work can proceed smoothly.

In the second embodiment, the selection operation for a new examination result via the second list 300 and the “NEXT” button images 400 serves as a trigger to start creating an examination report on the examination result that was selected right before (that is, displayed right before in the display area) (S27). The examination report is created in the background while subsequent examination results are being displayed in the display area 200. The examination reports are temporarily stored on a computer's memory (e.g., a temporary storage area, e.g., main memory).

These steps are repeated for the examination results that were collectively selected at once.

In addition, if an examination result that has already been displayed once is selected and displayed again before transferred, and further editing is performed on the examination result, the examination report that was created in advance is discarded and a new examination report may be created based on the edited (updated) examination result.

A transfer button image (a connecting button image) is displayed on the examination result display screen image. When the transfer button image 600 is touched (S28: YES), transfer of the examination report to the medical information system 30 is started. The examination report corresponding to at least a part of the plurality of examination results that were collectively selected at once is generated in advance. Therefore, the created examination report may be transferred first. The remaining examination reports that are not created when the transfer button 600 is touched will be created after the transfer button image 600 is touched (S29). The process of creating the remaining examination reports may be performed after the transfer button image 600 was touched but before the transfer is started.

Alternatively, the process of creating the remaining examination reports may be performed in parallel with transferring of the created examination reports. A transfer status may be grasped by the examiner via a progress bar or the like that is displayed. Furthermore, the viewer may be automatically closed after the transfer is completed. In this way, in this embodiment, at least some of the multiple examination reports are created before accepting the transfer operation. Therefore, it is possible to reduce a time after the transfer operation for the multiple examination reports is input until transfer of all the examination reports is completed.

### Modified Example

Although the embodiments have been described above, the present disclosure is not necessarily limited to the above embodiments.

For example, in the above embodiment, both the manual transfer and the automatic transfer are used, but only one of them may be implemented in the device.

For example, in the first embodiment, the automatic transfer was executed upon completion of the examination. However, the trigger for the automatic transfer is not necessarily limited to this, and for example, activation of the viewer may be the trigger for the automatic transfer. Alternatively, the automatic transfer trigger may be a timing at which the patient search (S1) is performed to examine a new subject (i.e., a new examinee).

For example, in the first embodiment, one examination report is transferred in response to each transfer operation, and in the second embodiment, a plurality of examination reports are transferred at once based on the single transfer operation. These two techniques may be used together. For example, in the second embodiment, two types of transfer operations: an individual transfer operation for individual transfer and a batch transfer operation for batch transfer may be set, and the two types of transfer operations may be selectively accepted. Then, when multiple examination results are selected in advance, the transfer may be performed in a selected way.

In addition, in each example, if the follow-up examination is performed and the examination report is transferred based on the transfer operation, the examination report based on the current examination results may be created under the same report creation conditions as the examination report based on the past examination results. In this case, the present examination report may be automatically created without requesting that the present examination result be displayed on the examination result display screen image. The report creation condition may be a condition that specifies at least one of the types of reports in the previous examination report and the editing result of the report. An appropriate examination report can be created based on the examination results obtained by the follow-up examination without the need for an examiner to confirm the results. The reports automatically created in this way may also be targets for the batch transfer.

### <Other Forms>

The present disclosure also discloses the following ophthalmology information processing program and ophthalmology information processing device.

A first ophthalmology information processing program for at least one processor of an ophthalmology computer, the program comprising instructions, when executed by the at least one processor, cause the at least one processor to:
acquire an examination result of a subject's eye examined by an eye examination device;
generate a converted examination result of the subject's eye based on the examination result and a template that was prepared in advance; and
selectively perform:
a first transfer step by (i) displaying the converted examination result, (ii) requesting for a transfer operation, and (iii) executing a transfer process on the converted examination result upon receiving the transfer operation; and a second transfer step by executing the transfer process on the converted examination result without requesting for the transfer operation.

In a second ophthalmological information processing program according to the first ophthalmological information processing program, the instructions further cause the at least one processor to perform, at the second transfer step, the transfer process on the converted examination result before displaying the converted examination result.

In a third ophthalmology information processing program according to the first or second ophthalmology information processing program, the instructions further cause the at least one processor to create a plurality of examination reports as the converted examination result. The examination reports are created based on various types of templates that are associated with at least one of a disease type and examination conditions for the eye examination device. Each of the examination reports is created based on a respective one of the templates. At an acquisition step, the examination result and information regarding either the disease type in the subject eye and the examination conditions for the subject eye are acquired. At the second transfer step, the examination reports based on some of the plurality of templates are selectively transferred to a medical information system based on the information.

In a third ophthalmology information processing program according to the third ophthalmology information processing program, a binocular report based on the examination result of both eyes and a monocular report based on the examination result of one eye are created. Therefore, the binocular report and the monocular report are selectively transferred based on the examination result and either one of first information on whether a binocular disease exists or second information on whether the examination result on both left and right eyes exists.

An ophthalmology computer executes any one of the first to fourth ophthalmological information processing programs.

1. A non-transitory, computer readable storage medium storing an ophthalmology information processing program for at least one processor of an ophthalmology computer, the program comprising instructions, when executed by the at least one processor, cause the at least one processor to:
sequentially display a plurality of examination results on a screen, the plurality of examination results being examination results on a subject's eye obtained by an eye examination device;
generate a plurality of converted examination results by converting the plurality of examination results in a predetermined format; and
collectively perform a transfer process on the plurality of converted examination results each corresponding to a respective one of the plurality of examination results upon receiving a transfer operation, wherein
when generating the plurality of converted examination results, the instructions further cause the at least one processor to generate at least one of the plurality of converted examination results before receiving the transfer operation.

2. The storage medium according to claim 1, wherein
the instructions further cause the at least one processor to generate the at least one of the plurality of converted examination results in a background while the plurality of examinations results are being sequentially displayed on the screen.

3. The storage medium according to claim 1, wherein
the instructions further cause the at least one processor to edit, based on an editing operation, a content of the converted examination result corresponding to the examination result that is currently displayed on the screen.

4. The storage medium according to claim 1, wherein
the instructions further cause the at least one processor to:
switch from the examination result that is currently displayed on the screen to another examination result upon receiving a switching operation; and
generate the converted examination result corresponding to the previous examination result that was displayed immediately before the current examination result is displayed based on the switching operation.

5. The storage medium according to claim 1, wherein
when generating the plurality of converted examination results, the instructions further cause the at least one processor to create at least one examination report on the subject's eye based on the plurality of examination results and a template that was prepared in advance.

6. The storage medium according to claim 1, wherein
the instructions further cause the at least one processor to selectively perform, when transferring the plurality of converted examination results:
a first transfer step by (i) sequentially displaying the plurality of examination results, (ii) requesting for the transfer operation, and (iii) performing the transfer process on the examination result for which the transfer operation is received; and
a second transfer step by performing the transfer process on the plurality of examination results without requesting for the transfer operation.

7. The storage medium according to claim 6, wherein
the instructions further cause the at least one processor to, at the second transfer step, perform the transfer process on the plurality of examination results before displaying the plurality of examination results.

8. The storage medium according to claim 6, wherein
the instructions further cause the at least one processor to:
when generating the plurality of converted examination results, create at least one report on the subject's eye based on the plurality of examination results and at least one of various types of templates that were prepared in advance, each of the templates being in association with at least one of a disease type and examination conditions for the eye examination device;
acquire the plurality of examination results and information on either the disease type of the subject's eye and the examination conditions; and
at the second transfer step, selectively transfer, based on the acquired information, the at least one examination report created based on the at least one of the various types of the templates.

9. The storage medium according to claim 8, wherein
the information includes at least one of first information on whether a binocular disease exists and second information on whether the plurality of examination results on both left and right eyes exist, and
the instructions further cause the at least one processor to:
when generating the plurality of converted examination results, create a binocular report based on the examination result on both eyes and a monocular report based on the examination result on one eye; and
selectively transfer the binocular report and the monocular report based on the first information or the second information.

10. An ophthalmology computer, comprising
at least one processor; and
at least one memory storing an ophthalmology information processing program, wherein
the at least one processor causes, by executing the program, the ophthalmology computer to:
sequentially display a plurality of examination results on a screen, the plurality of examination results being examination results of a subject's eye obtained by an eye examination device;
generate a plurality of converted examination results by converting the plurality of examination results in a predetermined format; and
collectively perform a transfer process on the plurality of converted examination results each corresponding to a respective one of the plurality of examination results upon receiving a transfer operation, wherein
when generating the plurality of converted examination results, the at least one processor further causes the ophthalmology computer to generate at least one of the plurality of converted examination results before receiving the transfer operation.

11. A method implemented by an ophthalmology computer, comprising:
sequentially displaying a plurality of examination results on a screen, the plurality of examination results being examination results of a subject's eye obtained by an eye examination device;
generating a plurality of converted examination results by converting the plurality of examination results in a predetermined format; and
collectively performing a transfer process on the plurality of converted examination results each corresponding to a respective one of the plurality of examination results upon receiving a transfer operation, wherein
when generating the plurality of converted examination results, the method further comprises generating at least one of the plurality of converted examination results before receiving the transfer operation.
